(19) United States
(12) Patent Application Publication
(10) Pub. No.: US 2020/0129095 A1
(43) Pub. Date: Apr. 30, 2020

(54) APPARATUS AND METHOD FOR ESTIMATING BIOLOGICAL COMPONENT

(71) Applicant: SAMSUNG ELECTRONICS CO., LTD., Suwon-si (KR)

(72) Inventors: So Young LEE, Daejeon (KR); Sang Kon BAE, Seongnam-si (KR)

(73) Assignee: SAMSUNG ELECTRONICS CO., LTD., Suwon-si (KR)

(21) Appl. No.: 16/418,455

(22) Filed: May 21, 2019

(30) Foreign Application Priority Data
Oct. 29, 2018 (KR) 10-2018-0129536

(51) Int. Cl. A61B 5/1455; A61B 5/145; A61B 5/00; A61B 5/1495; G16H 50/20

(52) U.S. Cl. CPC A61B 5/1455; A61B 5/14532; A61B 5/14546; G16H 50/20; A61B 5/7267; A61B 5/7275; A61B 5/1495; A61B 5/7203

(57) ABSTRACT

An apparatus for estimating a biological component may include: a spectrometer configured to measure a background spectrum from an object; and a processor configured to generate a virtual spectrum as training data by combining the background spectrum with a virtual biological component signal including noise, and generate a prediction model for estimating the biological component based on the training data.

### CROSS-REFERENCE TO RELATED APPLICATION(S)

This application is based on and claims priority from Korean Patent Application No. 10-2018-0129536, filed on Oct. 29, 2018, in the Korean Intellectual Property Office, the disclosure of which is incorporated herein by reference in its entirety.

### BACKGROUND

### 1. Field

The following description relates to an apparatus and method for non-invasively estimating biological components.

### 2. Description of the Related Art

Diabetes is a chronic disease that causes various complications and can be hardly cured, such that people with diabetes are advised to check their blood glucose regularly to prevent complications. In particular, when insulin is administered to control blood glucose, the blood glucose levels have to be closely monitored to avoid hypoglycemia and control insulin dosage. An invasive method of finger pricking is generally used to measure blood glucose levels. However, while the invasive method may provide high reliability in measurement, it may cause pain and inconvenience as well as an increased risk of disease infections due to the use of injection. Recently, research has been conducted on a method of non-invasively estimating biological components, such as blood glucose, by using a spectrometer without blood sampling.

### SUMMARY

In an aspect of the present disclosure, there is provided an apparatus for estimating a biological component, the apparatus including: a spectrometer configured to measure a background spectrum from an object; and a processor configured to generate a virtual spectrum as training data by combining the background spectrum with a virtual biological component signal including noise, and to generate a prediction model for estimating the biological component based on the obtained training data.

The spectrometer may include: a light source configured to emit light onto the object; and a detector configured to detect light scattered or reflected from the object.

The processor may generate the virtual biological component signal based on a unit biological component spectrum and a light travel path of a light that emitted from the spectrometer and collected by the spectrometer after passing through the object.

The processor may generate the virtual biological component signal by adding a random noise to the virtual biological component signal for each wavelength of the virtual biological component signal.

The processor may perform smoothing on the virtual biological component signal.

The processor may perform a first smoothing on the virtual biological component signal for each wavelength, and then may perform a second smoothing on the virtual biological component signal for each of a plurality of calibration times.

The processor may further obtain a virtual biological component value of the virtual biological component signal as the training data, and may train the prediction model based on the training data.

The processor may obtain the virtual biological component value by combining a reference biological component value that is measured by an external device at a time when the background spectrum is measured, with a virtual biological component variation used for generating the virtual spectrum.

The processor may obtain a plurality of virtual biological component variations based on a plurality of biological component change patterns, and generate the virtual spectrum based on the plurality of virtual biological component variations.

The plurality of biological component change patterns may be pre-defined for each user based on at least one of a user's gender and age, a range of biological component values, and medical history (e.g., information on whether a user has a disease related to a biological component).

The processor may combine the background spectrum with the virtual biological component signal by using Lambert-Beer's law.

Once the spectrometer measures a spectrum for estimating the biological component, the processor may estimate the biological component by applying the prediction model to the spectrum that is measured for estimating the biological component.

The biological component may include at least one of blood glucose, cholesterol, triglycerides, proteins, and uric acid.

In another aspect of the present disclosure, there is provided a method of estimating a biological component, the method including: measuring a background spectrum from an object; obtaining a virtual spectrum as training data by combining the background spectrum with a virtual biological component signal including noise; and generating a prediction model for estimating the biological component based on the training data.

The obtaining the virtual spectrum as the training data may include generating the virtual biological component signal based on a unit biological component spectrum and a light travel path of a light that is emitted to the object and then reflected or scattered from the object after passing though the object.

The obtaining the virtual spectrum as the training data may further include generating a virtual biological component signal including the noise by adding a random noise to the virtual biological component signal for each wavelength of the virtual biological component signal.

The obtaining of the virtual spectrum as the training data may further include performing smoothing on the virtual biological component signal.

The obtaining the virtual spectrum as the training data may include performing a first smoothing on the virtual biological component signal for each wavelength, and then performing a second smoothing on the virtual biological component signal for each of a plurality of calibration time.

The obtaining the virtual spectrum as the training data may include obtaining a virtual biological component value of the virtual biological component signal as training data; and the generating the prediction model may include training the prediction model based on the training data.

In addition, the method of estimating a biological component may further include, measuring a spectrum for estimating the biological component; and estimating the biological component by applying the prediction model to the spectrum measured for estimating the biological component.

### BRIEF DESCRIPTION OF THE DRAWINGS

FIG. 1 is a block diagram illustrating an apparatus for estimating a biological component according to an embodiment of the present disclosure.

FIG. 2 is a block diagram illustrating a measurer according to an embodiment of the present disclosure.

FIG. 3 is a block diagram illustrating a processor according to an embodiment of the present disclosure.

FIGS. 4A, 4B, 4C, and 4D are diagrams explaining an example of a method of obtaining a virtual spectrum.

FIG. 5 is a block diagram illustrating an apparatus for estimating a biological component according to another embodiment of the present disclosure.

FIG. 6 is a flowchart illustrating a method of estimating a biological component according to an embodiment of the present disclosure.

FIG. 7 is a diagram illustrating a wearable device according to an embodiment of the present disclosure.

FIG. 8 is a block diagram illustrating the wearable device of FIG. 7.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

Details of other embodiments are included in the following detailed description and drawings. Advantages and features of the present disclosure, and a method of achieving the same will be more clearly understood from the following embodiments described in detail with reference to the accompanying drawings. Throughout the drawings and the detailed description, unless otherwise described, the same drawing reference numerals will be understood to refer to the same elements, features, and structures.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as ‘part’ or ‘module’, etc., should be understood as a unit that performs at least one function or operation and that may be embodied as hardware, software, or a combination thereof.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

Hereinafter, embodiments of an apparatus and method for estimating a biological component will be described in detail with reference to the accompanying drawings.

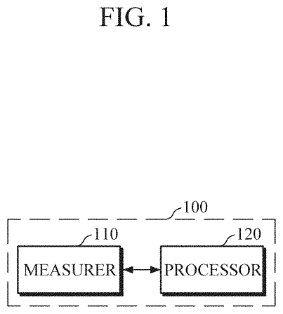

FIG. 1 is a block diagram illustrating an apparatus for estimating a biological component according to an embodiment of the present disclosure. The biological component estimating apparatus 100 according to an embodiment of the present disclosure may be embedded in a terminal such as a smartphone, a tablet PC, a desktop computer, a laptop computer, and the like, or a wearable device worn on an object. In this case, examples of the wearable device may include a wristwatch-type wearable device, a bracelet-type wearable device, a wristband-type wearable device, a ring-type wearable device, a glasses-type wearable device, a hairband-type wearable device, or the like, but the wearable device is not limited thereto, and may be embedded in a medical device manufactured for use in medical institutions to measure and analyze bio-information.

Referring to FIG. 1, the biological component estimating apparatus 100 includes a measurer 110 and a processor 120.

The measurer 110 measures a spectrum from an object. In particular, the object may be a user's skin (e.g., an upper portion of the wrist where veins or capillaries are located, or an area on the wrist that is adjacent to the radial artery). In the case where the area is the skin surface of the wrist where the radial artery passes, measurement may be relatively less affected by external factors, such as the thickness of a skin tissue in the wrist, which may cause errors in measurement. However, the object is not limited thereto, and may be a peripheral body part of the human body, such as fingers, toes, and the like, where blood vessels are densely located.

The measurer 110 may emit light onto an object according to a predetermined control signal, and may measure a spectrum by detecting light reflected or scattered from the object. In particular, the measurer 110 may measure a spectrum by using Infrared spectroscopy or Raman spectroscopy, but is not limited thereto, and may also use various spectroscopic methods. The measurer 110 may be realized as a spectrometer.

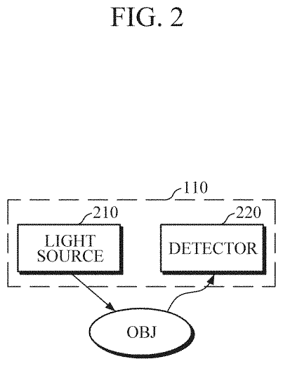

FIG. 2 is a block diagram illustrating a measurer according to an embodiment of the present disclosure. Referring to FIG. 2, the measurer 110 includes a light source 210 and a detector 220.

The light source 210 may include a light emitting diode (LED), a laser diode, a fluorescent body, and the like, and may emit light in a near-infrared ray (NIR) range or a mid-infrared ray (MIR) range. The light source 210 may be formed as an array of a plurality of light sources which emit light in various wavelength ranges to obtain accurate spectrum data. Once the light source 210 emits light onto a user's skin, which is an object OBJ, according to a control signal of the processor 120, the emitted light passes through the skin to arrive at a body tissue. Upon arriving at the body tissue, light is scattered or reflected from the body tissue and returns passing through the user's skin.

The detector 220 may measure a spectrum by detecting light which returns after passing through the user's skin. The detector 220 may include an InGaAs photodiode, and the like, and may be formed as an array of a plurality of detectors 220.

There may be various numbers and arrangements of the light source 210 and the detector 220, and the number and arrangement thereof may vary according to types and a purpose of use of a biological component, the size and shape of the wearable device, and the like.

Referring back to FIG. 1, the processor 120 processes various signals and operations associated with estimation of a biological component of an object. Here, the biological component may include blood components such as blood glucose, triglycerides, cholesterol, calories, proteins, uric acid, and the like, but is not limited thereto.

For example, in response to receipt of a user's request or in response to predetermined criteria being satisfied, the processor 120 may control the measurer 110 to measure a spectrum from an object. For example, in response to receipt of a user's request or in response to criteria for calibration being satisfied, the processor 120 may control the measurer 110 to measure a background spectrum for calibration for a reference period of time, (e.g., about one hour on an empty stomach). Alternatively, in response to receipt of a user's request for estimating a biological component, or in response to criteria for estimating a biological component being satisfied, the processor 120 may control the measurer 110 to measure a target spectrum for estimating a biological component from an object.

Once the measurer 110 measures the background spectrum, the processor 120 may generate a virtual spectrum by combining the background spectrum with a virtual biological component signal including noise. In particular, the processor 120 may combine the background spectrum with the virtual biological component signal including noise by using Lambert-Beer's law.

The processor 120 may define a virtual variation of a biological component concentration, and may generate a virtual biological component signal by combining the defined virtual variation of the biological component concentration with a pre-input unit biological component spectrum and a light travel path. Further, the processor 120 may acquire a virtual biological component signal including noise by adding a random noise to the generated virtual biological component signal. The random noise may refer to a random signal having equal intensity at different frequencies and having a constant power spectral density. An example of the random noise may include white noise. Upon adding the random noise to the virtual biological component signal, the processor 120 may acquire a virtual biological component signal including noise by performing various preprocessing operations such as smoothing and the like.

Here, the unit biological component spectrum may be pre-obtained by using a transmission method or a reflection method using a water solution, a phantom solution, a blood solution. The light travel path may be measured directly, or may be estimated by simulation or based on an absorption rate of a solution.

The processor 120 may generate a plurality of virtual spectra by defining the virtual variation of the biological component concentration as various values. The processor 120 may obtain the generated plurality of virtual spectra and a plurality of virtual biological component values, corresponding to the plurality of virtual spectra, as training data. The processor 120 may obtain a virtual biological component value by combining the virtual variation of the biological component concentration, which is used for generating the virtual spectrum, with a reference biological component value. In this case, the reference biological component value may be a biological component value measured using an external device for measuring a biological component at a time when a background spectrum is measured. Here, the external device for measuring a biological component may be an invasive/minimally invasive device for measuring a biological component, but is not limited thereto. Examples of the external device may include a blood glucose meter using a lancet and a test strip, and a urine glucose test kit.

In addition, the processor 120 may obtain a plurality of virtual variations of a biological component concentration based on a plurality of pre-defined change patterns of a biological component. For example, the processor 120 may pre-define a plurality of biological component change patterns for each user based on at least one of a user's gender and age, a range of biological component values, and medical history (e.g., information on whether a user has a disease related to a biological component).

The processor 120 may generate a prediction model for estimating a biological component based on the obtained training data. Further, once the measurer 110 measures a target spectrum for estimating a biological component, the processor 120 may estimate a biological component by applying a prediction model.

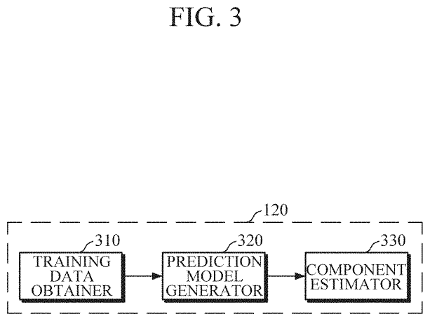

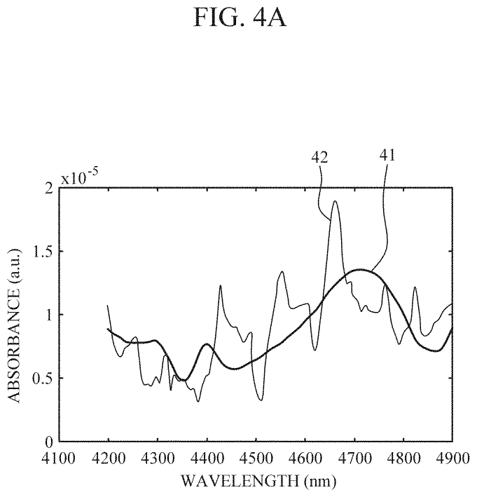

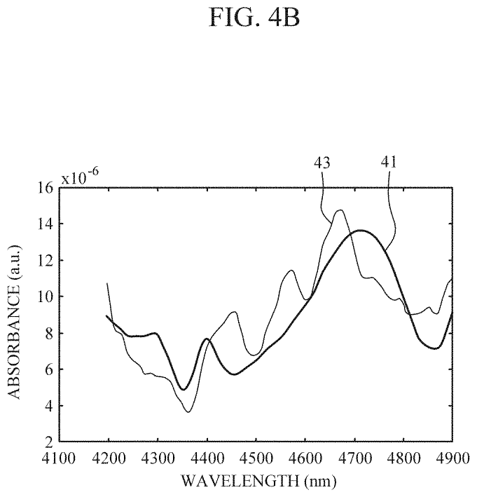

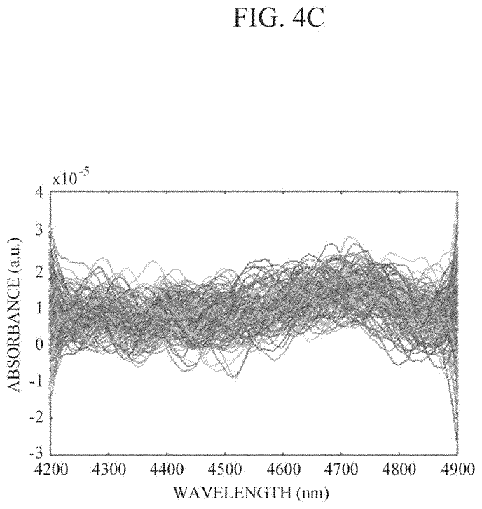

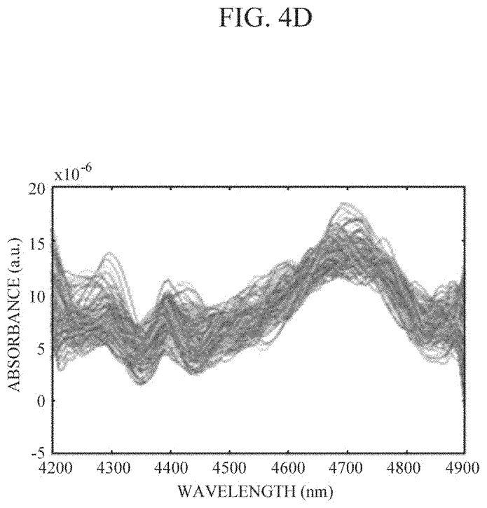

FIG. 3 is a block diagram illustrating a processor according to an embodiment of the present disclosure. FIGS. 4A to 4D are diagrams explaining an example of a method of obtaining a virtual spectrum. Referring to FIGS. 1 to 4D, an example of a processor 120 will be described below. Hereinafter, for convenience of explanation, description will be made using blood glucose as an example of a biological component.

Referring to FIG. 3, the processor 120 includes a training data obtainer 310, a prediction model generator 320, and a component estimator 330.

The training data obtainer 310 may obtain a plurality of virtual spectra and virtual blood glucose values, corresponding to the plurality of virtual spectra, as training data for training a prediction model

The training data obtainer 310 may define a virtual variation of a blood glucose concentration by considering various blood glucose change patterns. A variation of a blood glucose concentration may indicate a deviation from an average blood glucose concentration during a given time period. Examples of the variation may include a standard deviation (SD) and a coefficient of variation (CV). The various blood glucose change patterns may be defined to be commonly applicable to all users, or may be defined for each user or according to age or gender characteristics of users. For example, the training data obtainer 310 may define a range or a pattern of virtual variations of the blood glucose concentration based on a minimum blood glucose value or a maximum blood glucose value which are on hourly, daily, weekly, and seasonal bases for each user. In another example, the training data obtainer 310 may also define variations of the blood glucose concentration differently according to information whether a user is a diabetic patient. For example, the training data obtainer 310 may define a first virtual blood glucose concentration variation for minors with type 1 diabetes, a second virtual blood glucose concentration variation for minors with type 2 diabetes, a third virtual blood glucose concentration variation for minors without diabetes, a fourth virtual blood glucose concentration variation for adults with type 1 diabetes, a fifth virtual blood glucose concentration variation adults with type 2 diabetes, and a sixth virtual blood glucose concentration variation adults without diabetes. However, the training data obtainer 310 is not limited to these examples, and may be modified in various manners.

Upon defining various virtual variations of the blood glucose concentration, the training data obtainer 310 may obtain a virtual spectrum and a virtual blood glucose value by using a pre-defined equation. The training data obtainer 310 may obtain the virtual spectrum and the virtual blood glucose value at each of a plurality of times during a predetermined period of time (e.g., about one hour) when a background spectrum for calibration is measured.

For example, the following Equation 1 represents an equation for obtaining a virtual spectrum (FS) defined based on Lambert-Beer's law. However, the equation is not limited thereto.

FS=BS+ε_{g}·ΔCₜ+N_{g} [Equation 1]

Herein, BS denotes the background spectrum measured from a user of the biological component estimating apparatus 100 or at least one human test subject, at a reference time, (e.g., a time of an empty stomach), for example, while the user or the human test subject is in a fasting state. The background spectrum may include information about a blood glucose component on an empty stomach, and noise. ε_{g} denotes the unit blood glucose spectrum, and L denotes a length of the light travel path of the light emitted from the light source 210. The light travel path may indicate a distance that the light travels through a sample (e.g., the user or the human test subject), starting from the light source 210 to the detector 220. In this case, the unit blood glucose spectrum and the light travel path are pre-input values. ΔCₜ denotes the defined virtual variation of the blood glucose concentration. ε_{g}·L·ΔCₜ denotes the blood glucose component signal, and Ng denotes the random noise added for each wavelength.

The following Equation 2 represents an equation for obtaining a virtual blood glucose value.

Cₜ=ΔCₜ+C₀ [Equation 2]

Herein, C₀ denotes a reference blood glucose value, and a blood glucose value at a time of calibration (e.g., a time of an empty stomach), and Cₜ denotes a virtual blood glucose value. It may be assumed that a blood glucose value C₀ on an empty stomach is constant, such that by defining the virtual blood glucose variation ΔCₜ, the virtual blood glucose value Cₜ may be obtained using Equation 2.

FIG. 4A illustrates, at a time T, a virtual blood glucose signal 41 (hereinafter referred to as a “first virtual blood glucose signal”) including a pure glucose concentration, and a virtual blood glucose signal 42 (hereinafter referred to a “second virtual blood glucose signal”) generated by adding a random noise to the first virtual blood glucose signal for each wavelength. FIG. 4B illustrates a virtual blood glucose signal 43 (hereinafter referred to as a “third virtual blood glucose signal”) obtained by smoothing the second virtual blood glucose signal 42 of FIG. 4A for each wavelength.

As illustrated in FIG. 4A, the training data obtainer 310 may define the pure glucose concentration as a virtual blood glucose variation, and may generate the first virtual blood glucose signal 41 by combining the defined virtual blood glucose variation and the light travel path, as described above. Then, the training data obtainer 310 may generate the second virtual blood glucose signal 42 by adding a random noise to the first virtual blood glucose signal 41 for each wavelength thereof.

Further, as illustrated in FIG. 4B, the training data obtainer 310 may obtain the third virtual blood glucose signal 43 by smoothing the second virtual blood glucose signal 42 for each wavelength, so that absorbance of the second virtual blood glucose signal 42 may become similar to absorbance of the first virtual blood glucose signal 41.

In addition, the training data obtainer 310 may obtain the third virtual blood glucose signal 43 at a plurality of measurement times during a predetermined period of time, e.g., a period of time when a background spectrum is measured for calibration. The training data obtainer 310 may obtain a plurality of different second virtual blood glucose signals 42 by defining glucose concentrations, which are different at each measurement time, as virtual blood glucose variations, or by adding different random noises to the first virtual blood glucose signal 41 including the same virtual blood glucose variation.

FIG. 4C illustrates a plurality of third virtual blood glucose signals obtained by performing smoothing on the second virtual blood glucose signals for each wavelength, which are generated at each of a plurality of times 1 to T during a predetermined period of time. The plurality of times may refer to a plurality of calibration times that are applied to Equation 2 for calculating corresponding virtual blood glucose values. FIG. 4D illustrates a virtual blood glucose signal (hereinafter referred to as a “fourth virtual blood glucose signal”) obtained by performing smoothing on a plurality of third virtual blood glucose signals of each of the times 1 to T at each wavelength with respect to a time t after smoothing the third virtual blood glucose signals for each wavelength as illustrated in FIG. 4C.

As illustrated in FIG. 4C, the second virtual blood glucose signals of each of the times 1 to T are generated by adding an irregular random noise for the same wavelength of the first virtual blood glucose signal of each of the times 1 to T, such that patterns of the third virtual blood glucose signals may be irregular, which are obtained by separately smoothing the second virtual blood glucose signals of each of the times 1 to T for each wavelength. Accordingly, as illustrated in FIG. 4D, the training data obtainer 310 may obtain the fourth virtual blood glucose signal by performing smoothing on the third virtual blood glucose signals at each wavelength with respect to the time t. As illustrated in FIG. 4D, the obtained fourth virtual blood glucose signal of each of the times 1 to T may have a similar pattern for each wavelength.

Upon obtaining the fourth virtual blood glucose signal at the plurality of times as described above, the training data obtainer 310 may obtain a virtual spectrum by combining the background spectrum with each fourth virtual blood glucose signal, as represented by the above Equation 1.

In addition, the training data obtainer 310 may generate a virtual blood glucose value by adding a reference blood glucose value to the virtual blood glucose variation, which is used for obtaining each virtual spectrum at a plurality of times as represented by the above Equation 2, and may obtain the generated virtual blood glucose value of each time as training data.

The prediction model generator 320 may generate a prediction model for estimating a biological component by training a prediction model using the training data obtained by the training data obtainer 310. The prediction model generator 320 may generate a prediction model, which represents a correlation between an actually measured spectrum and an estimated blood glucose value, by training a prediction model having a virtual spectrum as an independent variable and a virtual blood glucose value as a dependent variable. For example, the prediction model generator 320 may train a prediction model by using various techniques such as a regression algorithm, artificial intelligence, and the like. For example, examples of the regression algorithm may include linear regression, non-linear regression, partial least squares, neural network regression, support vector regression, decision tree based regression. Naive Bayes regression, and the like, but the regression algorithm is not limited thereto.

The following Equation 3 represents a prediction model generated by training in the form of an equation. Equation 3 is an example of a simple linear function, but is not limited thereto.

y=ax+b [Equation 3]

Herein, y denotes an estimated blood glucose value; x denotes a target spectrum measured for estimating blood glucose; and a and b denote constants determined by training.

The component estimator 330 may estimate blood glucose by using the generated prediction model. Once the measurer 110 measures a target spectrum for estimating blood glucose in response to a user's request for estimating blood glucose or according to predetermined criteria, the component estimator 330 may estimate a blood glucose value by inputting the measured target spectrum into the prediction model.

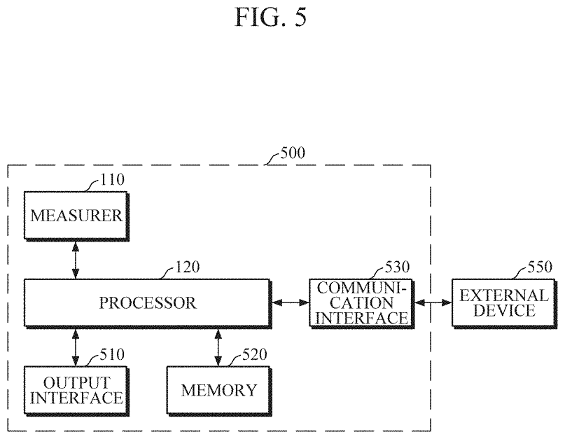

FIG. 5 is a block diagram illustrating an apparatus for estimating a biological component according to another embodiment of the present disclosure.

Referring to FIG. 5, the biological component estimating apparatus 500 includes the measurer 110, the processor 120, an output interface 510, a memory 520, and a communication interface 530. The measurer 110 and the processor 120 are described above in detail with reference to FIGS. 1 to 4D, such that the following description will be made based on other parts.

The output interface 510 may output results processed by the measurer 110 and/or the processor 120. For example, the output interface 510 may visually output an estimated biological component value by using a display module, or may output the value in a non-visual manner through voice, vibrations, tactile sensation, and the like by using a speaker module, a haptic module, and the like. The output interface 510 may divide a display area into two or more areas according to a predetermined setting, in which the output interface 510 may output a graph regarding a background spectrum and/or a virtual spectrum, which are used for estimating a biological component, or an estimated biological component value in a first area; and may output a biological component estimation history in the form of graphs and the like in a second area. In this case, if an estimated biological component value falls outside a normal range, the output interface 510 may output warning information in various manners, such as highlighting an abnormal value in red and the like, displaying the abnormal value along with a normal range, outputting a voice warning message, adjusting a vibration intensity, and the like.

The memory 520 may store processing results of the measurer 110 and/or the processor 120. Further, the memory 520 may store various criteria required for estimating a biological component. For example, the criteria may include user feature information such as a user's age, gender, a maximum value and/or a minimum value of a biological component, a health condition, and the like. In addition, the criteria may include a reference bio-signal value, a prediction model, a bio-signal estimation interval, a calibration interval, and the like, but are not limited thereto.

In this case, the memory 520 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The communication interface 530 may communicate with an external device 550 by using wired or wireless communication techniques under the control of the processor 120, and may transmit and receive various data to and from the external device 550. For example, the communication interface 530 may transmit a bio-signal estimation result to the external device 550, and may receive various criteria required for estimating a bio-signal from the external device 550. In this case, examples of the external device 550 may include an invasive/minimally invasive device for measuring a bio-signal, an information processing device such as a smartphone, a tablet PC, a desktop computer, a laptop computer, and the like.

In this case, examples of the communication techniques may include Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G communication, 4G communication, 5G communication, and the like. However, this is merely exemplary and is not intended to be limiting.

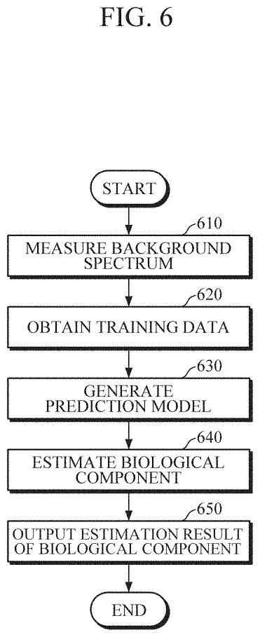

FIG. 6 is a flowchart illustrating a method of estimating a biological component according to an embodiment of the present disclosure. The method of estimating a biological component of FIG. 6 may be an example of a biological component estimating method performed by the biological component estimating apparatuses 100 and 500 according to the embodiments of FIGS. 1 and 5, which is described above in detail such that description thereof will be briefly made below in order to avoid redundancy.

Referring to FIG. 6, the biological component estimating apparatuses 100 and 500 may measure a background spectrum for calibration in operation 610. For example, when a user is on an empty stomach, the biological component estimating apparatuses 100 and 500 may measure a background spectrum from a skin tissue of the user for a predetermined period of time. It is assumed that a biological component, e.g., a blood glucose value, is constant on an empty stomach, but the embodiment is not limited thereto.

Then, the biological component estimating apparatuses 100 and 500 may obtain training data based on the background spectrum in operation 620. For example, the biological component estimating apparatuses 100 and 500 may generate a virtual spectrum by combining the background spectrum with a virtual biological component signal including noise based on Lambert-Beer's law, and may obtain the generated virtual spectrum as training data.

For example, the biological component estimating apparatuses 100 and 500 may define a virtual variation of a biological component by considering various biological component change patterns at each time during a predetermined period of time, e.g., a period of time when the background spectrum is measured in operation 610, and may generate a virtual biological component signal by combining the virtual biological component variation defined for each time with a unit biological component spectrum and a light travel path. The biological component estimating apparatuses 100 and 500 may add a random noise to the generated virtual biological component signal of each time for each wavelength, and may obtain the virtual biological component signal including the noise for each time.

The biological component estimating apparatuses 100 and 500 may first perform smoothing on the virtual biological component signals for each wavelength, which are obtained for each time and include the random noise, and then may perform smoothing on the virtual biological component signals at each wavelength with respect to each time. The biological component estimating apparatuses 100 and 500 may obtain a virtual spectrum by combining the background spectrum with the virtual biological component signal obtained by smoothing for each time and each wavelength, and including the noise.

Further, the biological component estimating apparatuses 100 and 500 may generate a virtual biological component value by combining the virtual biological component variation, defined for each time, with a reference biological component value, e.g., a biological component value measured on an empty stomach, and may obtain the generated virtual biological component value as training data.

Then, upon obtaining the training data in operation 620, the biological component estimating apparatuses 100 and 500 may generate a prediction model by using the obtained training data in operation 630. For example, the biological component estimating apparatuses 100 and 500 may generate a prediction model using a spectrum as an independent variable and an estimated biological component value as a dependent variable, and may train a prediction model by using the plurality of virtual spectra and virtual biological component values obtained as training data.

Subsequently, upon measuring a target spectrum for estimating a biological component in response to a user's request or according to predetermined criteria, the biological component estimating apparatuses 100 and 500 may estimate a biological component in operation 640 by applying the prediction model generated in operation 630.

Next, the biological component estimating apparatuses 100 and 500 may output an estimation result of a biological component in operation 650. For example, the biological component estimating apparatuses 100 and 500 may provide the estimation result of a biological component to a user by various visual methods using visual output devices, such as a display and the like, or by non-visual methods through vibrations, tactile sensation, and the like using a speaker module and/or a haptic module.

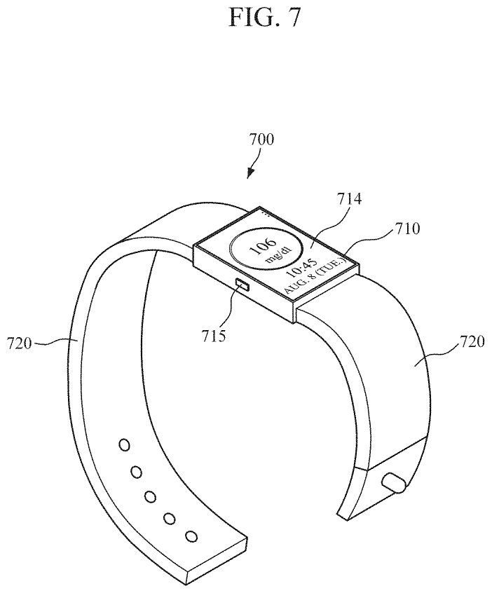

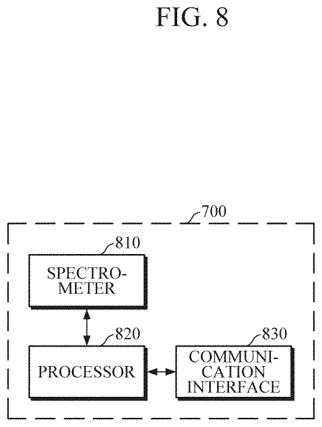

FIG. 7 is a diagram illustrating a wearable device according to an embodiment of the present disclosure. FIG. 8 is a block diagram illustrating the wearable device of FIG. 7. Various embodiments of the biological component estimating apparatuses 100 and 500 described above may be mounted in a smart band-type wearable device or a smart watch-type wearable device. However, the wearable device is merely an example for convenience of explanation, and the biological component estimating apparatuses are not limited thereto, and may be mounted in various information processing devices such as a smartphone, a tablet PC, a desktop computer, and the like.

Referring to FIGS. 7 and 8, the wearable device 700 includes a main body 710 and a strap 720.

The strap 720 may be flexible, and may be connected to both ends of the main body 710 to be bent around a user's wrist or may be bent in a manner which allows the strap 720 to be detached from a user's wrist. In this case, a battery may be embedded in the main body 710 or the strap 720 to supply power to various modules of the wearable device 700.

The main body 710 of the wearable device 700 may include a spectrometer 810 which emits light onto skin of a user, and measures a spectrum by detecting light scattered or reflected from the skin; and a processor 820 which estimates a biological component of a user by using the spectrum measured by the spectrometer 810.

The spectrometer 810 may emit light onto skin of a user's wrist by driving a light source according to a control signal of the processor 820, and may detect light returning from the skin of the user. Light emitted by the light source passes through the skin to arrive at a body tissue, and upon arriving at the body tissue, the light reacts with the body tissue to be absorbed thereinto, or scattered or reflected therefrom. The spectrometer 810 obtains the scattered or reflected light as a spectrum, and transmits the obtained spectrum to the processor 820. In this case, the light source may be configured to emit light in a near-infrared range or a mid-infrared range.

The spectrometer 810 may include a Linear Variable Filter (LVF). The LVF has spectral properties which vary linearly over the entire length. Accordingly, the LVF may scatter the incident light in order of wavelengths. Although having a compact size, the LVF has excellent light scattering ability.

A display 714 may be mounted on a front surface of the main body 710 of the wearable device 700 to provide various types of information to a user. The display 714 may include a touch panel which allows touch input of a user, and may receive a touch input from a user and transmit the received input to the processor 820.

Further, the main body 710 of the wearable device 700 may include a manipulator 715 which allows a user to input a command for controlling various functions of the wearable device 700. The manipulator 715 may be electrically connected to the processor 820, and may transmit various control commands of a user to the processor 820. Further, the manipulator 715 may include a power button for inputting a command to turn on/off the wearable device 700.

The processor 820 may process various commands input from a user through the display 714 and/or the manipulator 715. For example, upon receiving a command for calibration from a user, the processor 820 may perform calibration for generating a prediction model. The processor 820 may control the spectrometer 810 to measure a background spectrum for calibration. As described above, upon measuring the background spectrum, the processor 820 may obtain a plurality of virtual spectra and virtual biological component values as training data, and may generate a final prediction model for estimating a biological component by training the prediction model using the training data.

The processor 820 may store the final prediction model, generated by calibration, in a storage device, and may use the stored final prediction model later for estimating a biological component. In this case, the storage device may be mounted in the main body 710 of the wearable device 700, or may be mounted in an external device which may be connected by wire or wirelessly to the wearable device 700.

Upon receiving a command for estimating a biological component from a user, the processor 820 may control the spectrometer 810 to measure a target spectrum for estimating a biological component. Once the spectrometer 810 measures the target spectrum, the processor 820 may estimate a biological component by applying the prediction model stored in the storage device to the target spectrum.

The processor 820 may control the display 714 to display the generated various types of information, e.g., information associated with a biological component, or various other types of information (e.g., time, weather, etc.), to provide the information to a user.

The communication interface 830 may communicate with an external device under the control of the processor 820, in which examples of the external device may include not only an invasive apparatus for measuring a biological component, but also an information processing device such as a mobile terminal, a desktop computer, a laptop computer, and the like of a user.

While not restricted thereto, an example embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an example embodiment may be written as a computer program transmitted over a computer-readable transmission medium, such as a carrier wave, and received and implemented in general-use or special-purpose digital computers that execute the programs. Moreover, it is understood that in example embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for estimating a biological component, the apparatus comprising:
a spectrometer configured to measure a background spectrum from an object; and
a processor configured to generate a virtual spectrum as training data by combining the background spectrum with a virtual biological component signal including noise, and generate a prediction model for estimating the biological component based on the training data.

2. The apparatus of claim 1, wherein the spectrometer comprises:
a light source configured to emit light onto the object; and
a detector configured to detect light scattered or reflected from the object.

3. The apparatus of claim 1, wherein the processor is further configured to generate the virtual biological component signal based on a unit biological component spectrum and a light travel path of a light that emitted from the spectrometer and collected by the spectrometer after passing through the object.

4. The apparatus of claim 3, wherein the processor is further configured to generate the virtual biological component signal by adding a random noise to the virtual biological component signal for each wavelength of the virtual biological component signal.

5. The apparatus of claim 4, wherein the processor is further configured to perform smoothing on the virtual biological component signal.

6. The apparatus of claim 4, wherein the processor is further configured to perform a first smoothing on the virtual biological component signal for each wavelength, and then performs a second smoothing on the virtual biological component signal for each of a plurality of calibration times

7. The apparatus of claim 1, wherein the processor is further configured to obtain a virtual biological component value of the virtual biological component signal as the training data, and train the prediction model based on the training data.

8. The apparatus of claim 7, wherein the processor is further configured to obtain the virtual biological component value by combining a reference biological component value that is measured by an external device at a time when the background spectrum is measured, with a virtual biological component variation used for generating the virtual spectrum.

9. The apparatus of claim 1, wherein the processor is further configured to obtain a plurality of virtual biological component variations based on a plurality of biological component change patterns, and generate the virtual spectrum based on the plurality of virtual biological component variations.

10. The apparatus of claim 9, wherein the plurality of biological component change patterns are pre-defined for each user based on at least one of a user's gender, age, and medical history.

11. The apparatus of claim 1, wherein the processor is further configured to combine the background spectrum with the virtual biological component signal by using Lambert-Beer's law.

12. The apparatus of claim 1, wherein the spectrometer is further configured to measure a spectrum for estimating the biological component, the processor estimates the biological component by applying the prediction model to the spectrum that is measured for estimating the biological component.

13. The apparatus of claim 1, wherein the biological component comprises at least one of blood glucose, cholesterol, triglycerides, proteins, and uric acid.

14. A method of estimating a biological component, the method comprising:
measuring a background spectrum from an object;
obtaining a virtual spectrum as training data by combining the background spectrum with a virtual biological component signal including noise; and
generating a prediction model for estimating the biological component based on the training data.

15. The method of claim 14, wherein the obtaining the virtual spectrum as the training data comprises generating the virtual biological component signal based on a unit biological component spectrum and a light travel path of a light that is emitted to the object and then reflected or scattered from the object after passing though the object.

16. The method of claim 15, wherein the obtaining the virtual spectrum as the training data further comprises generating the virtual biological component signal by adding a random noise to the virtual biological component signal for each wavelength of the virtual biological component signal.

17. The method of claim 16, wherein the obtaining the virtual spectrum as the training data further comprises performing smoothing on the virtual biological component signal.

18. The method of claim 14, wherein the obtaining the virtual spectrum as the training data comprises performing a first smoothing on the virtual biological component signal for each wavelength, and then performing a second smoothing on the virtual biological component signal for each of a plurality of calibration times.

19. The method of claim 14, wherein:
the obtaining the virtual spectrum as the training data comprises obtaining a virtual biological component value of the virtual biological component signal as the training data; and
the generating the prediction model comprises training the prediction model based on the training data.

20. The method of claim 14, further comprising: measuring a spectrum for estimating the biological component; and estimating the biological component by applying the prediction model to the spectrum measured for estimating the biological component.
